(19) United States
(12) Patent Application Publication
(10) Pub. No.: US 2015/0335475 A1
(43) Pub. Date: Nov. 26, 2015

(54) Device for dispensing drops

(71) Applicant: TECHNIPLAST, Louviers (FR)

(72) Inventors: Jean-Philippe LAMBOUX, Saint Didier des Bois (FR); Yvan POULLAIN, Pintherville (FR)

(21) Appl. No.: 14/721,232

(22) Filed: May 26, 2015

(30) Foreign Application Priority Data
May 26, 2014 (FR) 14 54738

(51) Int. Cl. A61F 9/00; B05B 11/04

(52) U.S. Cl. CPC A61F 9/0008; B05B 11/047

(57) ABSTRACT

A device for dispensing drops of a liquid or semi-liquid product, includes:
a tube (12) which includes a deformable outer casing defining an internal space (16) containing the product and an opening; and
a drop-generating tip (14) which is arranged at the opening and is able to form and expel a drop of product from the tip following pressure applied to the outer casing, and to prevent any return of product into the tip when the pressure on the casing ceases.

The invention relates to a device for dispensing a liquid or semi-liquid product in the form of drops.

Devices known to date for generating drops of product are found for example in the field of ophthalmic products and are divided into two categories.

The first category includes single-dose devices of which the container, made of flexible plastic (the containers are generally manufactured and offered for sale in the form of a package containing a strip of containers which are fixed to one another in a way such that a user can detach them), is sealed by a stopper that has to be detached irreversibly prior to first use. The user presses firmly against the outer casing of the container in order to expel the liquid product in the form of drops via the non-resealable orifice. This type of device is designed to deliver a single dose of product (a daily dose made up of one or more drops) and as soon as the user has extracted the prescribed dose from the container, even if some product remains inside, it is not possible to re-close the orifice and the product is therefore liable to deteriorate on contact with the air. The cost of the prescribed dose is therefore relatively expensive because it requires one container per dose.

The second category includes devices which take the form of a bottle containing several doses for a complete treatment over the course of several days. However, the product contained in the bottle is in contact with the ambient air each time the bottle is opened and is therefore exposed to the various contaminants carried in the air. As a result, manufacturers are forced to add to the product preservatives such as bactericides, antioxidants, etc. However, such additives are likely to give rise to reactions with the users' eyes.

Given the foregoing, there is therefore a need for a new device for dispensing drops of a liquid or semi-liquid product which overcomes at least one of the aforementioned disadvantages and notably which limits the contamination of the device and can find numerous applications including notably the field of ophthalmics.

Document US 2013/0037574 discloses a device for dispensing drops of liquid product in the form of doses, but which is not, however, satisfactory.

Thus one subject of the present invention is a device for dispensing drops of a liquid or semi-liquid product, characterized in that it comprises:
a tube which comprises, on the one hand, a deformable outer casing defining an internal space containing the product and, on the other hand, an opening,
a drop-generating tip which is arranged at the opening and is able, on the one hand, to form and expel a drop of product from the tip following pressure applied to the outer casing, and which tends to deform it and reduce the volume of the internal space and, on the other hand, to prevent any return of product into the tip when the pressure on the casing ceases, the drop-generating tip comprising an orifice through which a drop forms as the product passes through said orifice as a result of a pressure applied to the outer casing, the orifice being delimited at its periphery by at least one deformable wall which, on deforming, allows a drop to be formed,
a protective stopper which is fixed removably to the device to cover the drop-generating tip when the device is not being used, the protective stopper comprising an internal face which is directed to face the drop-generating tip when the protective stopper is fixed to the device, the internal face of the protective stopper being provided with at least one element made of absorbent material intended to absorb a drop at least partially formed on the outside of the orifice and which has not detached from this orifice, said at least one element made of absorbent material being distant from the orifice.

Such a device is capable of generating a single drop of product or, consecutively, several consecutive drops of product if the deformable outer casing is simply pressed several times or for a prolonged period of time. Once a drop has been formed by the drop-generating tip on the outside thereof, and when the action of pressing on the casing ceases, the drop thus formed can no longer return inside the tip (the tip thus acts as a check valve which opens and closes according to whether or not pressure is applied to the casing). Thus, ambient air therefore cannot enter the tip and the tube and contaminate the product remaining in this tube. The remaining product is thus protected from the outside and can be kept in the tube over a period ranging beyond several days without the need to add to the product the various agents such as preservatives, bactericides, antioxidants, etc. The product is therefore simpler to manufacture and in the users' eyes has the impression of being more “healthy” because it contains fewer ingredients.

The dose of product supplied by the device according to the invention therefore works out less expensive to dispense than with a single-dose device of the prior art. The tube can be a standard commercially available tube.

Moreover, arranging said at least one element made of absorbent material some distance from the orifice of the tip allows said at least one element made of absorbent material not to touch said at least one deformable wall and notably the orifice (end of the check valve), thus avoiding contaminating this zone.

It will be noted that the drop partially formed on the outside of the tip (where the tip is facing downwards and the orifice closes) is not heavy enough to counteract the surface tension which keeps it attached to the orifice. The drop therefore remains stuck to the orifice. The aforementioned arrangement thus allows the surplus product formed on the outside of the tip and adhering thereto to be absorbed, without contact with the tip, thereby avoiding any contamination of the tip. It will be noted that said at least one element made of absorbent material is positioned a distance away from the orifice that allows it to come into contact with the surplus product adhering to the orifice or to the wall surrounding same. Absorption is thus achieved by capillarity through the mere contact of said at least one element made of absorbent material with the external product surplus.

Said at least one element made of absorbent material is, for example, kept in a housing provided on the internal face of the stopper. The housing is, for example, produced from one or more elements which project from this face and away from the latter (parallel to the height or axial dimension of the stopper), this or these elements forming an external wall delimiting the housing. The transverse dimension of said at least one element made of absorbent material (perpendicular to the height of the stopper) is, for example, slightly greater than the transverse dimensions of the housing so that it can be introduced into the latter and held in place therein without any specific mechanical or chemical fastening. It is thus possible for said at least one element made of absorbent material to be removed with ease so that it can be replaced with another.

It will be noted that said at least one wall is said to be deformable in so far as it deforms elastically under the action of a stress (pressure) and returns to its non-deformed initial shape when the stress ceases. This property allows said at least one wall to deform repeatedly in this way over the course of time without becoming damaged.

According to other features considered in isolation or in combination with one another:
the drop-generating tip comprises means of plugging the orifice which are able to allow the orifice to open following a pressure applied to the outer casing and, in the absence of such pressure, to plug the orifice;
said at least one deformable wall is made of a flexible material;
said at least one flexible wall is able to close back:
either around a rigid member (e.g.: a valve needle) in the absence of pressure on the outer casing and to move away from the rigid member following pressure applied to the outer casing (the orifice is open and the rigid member is in the orifice),
or on the orifice in the absence of pressure on the outer casing (the orifice is thus closed when said at least one wall is closed again);
the device has no means of pumping the product;
the tube has a bottom which is positioned in a zone of the tube that is opposite the zone in which the opening is positioned, the tube being filled with product from the bottom;
the device is able to dispense a drop only when the tube is positioned with the drop-generating tip facing downward, which is particularly well suited to certain applications such as the dispensing of eye drops;
the internal space of the tube is partially filled with product so as therein to create a predetermined free volume which is greater than the volume of product contained in the non-deformable zone of the device, the predetermined free volume being occupied by a gas;
the internal face of the protective stopper is configured to press against said at least one deformable wall and prevent it from being deformed while the protective stopper is fixed to the device; when the internal face of the stopper (at least part thereof) is positioned or pressed against said at least one deformable wall (the latter forming one or more lips around the orifice and which, on deforming under the action of the pressure applied to the casing, open or enlarge the orifice), it prevents any deformation of said at least one deformable wall which might tend to open the orifice (if the orifice is closed at rest) or to enlarge it (if it is already open at rest and resting for example on a valve needle); this arrangement makes it possible for the device equipped with the stopper to be made fluid tight in a simple and reliable way without generating contamination of the tip; the internal face of the protective stopper is, for example, configured in a zone surrounding said at least one element made of absorbent material equipping a portion of the internal face of the stopper; the shape or configuration of the internal face of the stopper (or of part thereof) that allows said at least one deformable wall to be kept in or urged into its non-deformed or rest position may adopt various aspects and, for example, take on the appearance of one or more extensions of a part of this internal face away from the latter (it will be noted that this or these extensions may correspond to the element or elements forming the wall of the housing that receives said at least one element made of absorbent material); the extension may be axial (perpendicular to that part of the internal face of the stopper that is perpendicular to the axis of the tube when the stopper is on the tube) or may adopt other orientations (lateral or radial); it will be noted that the aforementioned extension or extensions are generally arranged around said at least one element made of absorbent material;
the protective stopper is pierced with a hole open to the outside of the device; this allows the zone internal to the stopper and situated behind the hole to be ventilated;
said at least one element made of absorbent material is arranged between the through-hole and the drop-generating tip so as to allow the product absorbed by said at least one element made of absorbent material to evaporate; said at least one element made of absorbent material thus communicates with the outside of the device equipped with its stopper so as to evaporate to the outside in the product that has been absorbed;
said at least one element made of absorbent material is positioned facing the orifice of the tip;
the device as briefly described hereinabove is applied to the dispensing of eye drops.

Another subject of the invention is a method for dispensing drops of a liquid or semi-liquid product from a device, characterized in that the device comprises:
a tube comprising, on the one hand, a deformable outer casing defining an internal space containing the product and, on the other hand, an opening,
a drop-generating tip which is arranged at the opening and comprises an orifice which is delimited at its periphery by at least one deformable wall,
   when the tube is positioned with the drop-generating tip facing downwards, the method comprising at least the following steps i) and ii):
i) application of an external pressure to the deformable outer casing of the device so as to deform it and reduce the volume of the internal space so as, on the one hand, to form at least one drop of product following deformation of said at least one deformable wall and the product passing through the orifice of the tip and, on the other hand, to expel said at least one drop thus formed from the tip,
ii) ceased application of an external pressure to the deformable outer casing of the device, the product situated outside the orifice of the tip being prevented from returning into the tip as a result of the reverse deformation of said at least one deformable wall and of the at least partial re-closure of the orifice, the product situated outside the orifice thus being suspended from the orifice,
iii) fitting of a protective stopper onto the device so as to cover the tip, the stopper comprising at least one element made of absorbent material which is distant from the orifice when the stopper is fixed on the device and which, through contact with the product suspended from the orifice on the outside thereof, absorbs said product.

An undispensed surplus of product may be present once the pressure on the casing ceases, said at least one deformable wall has returned to its initial (non-deformed) position and the orifice has closed again at least in part (which means to say that the size of the orifice has returned to its initial size, namely that the orifice has partially or fully closed again as the case may be), thus preventing any return of product into the tip and any further expelling of product. The fitting of a protective stopper comprising, held inside said stopper, at least one element made of absorbent material means that a product surplus remaining on the outside of the orifice can be absorbed. This makes it possible to avoid leaving product on the orifice (or said at least one deformable wall) to become contaminated by the surrounding air and thus also contaminates said at least one deformable wall surrounding the orifice. The fitting of the stopper can be done with the tip facing downward or upward. The method comprises the same advantages as the device set out hereinabove.

According to other features considered in isolation or in combination with one another:
step i) is repeated over the course of time so as to dispense several drops of product;
the series of steps i) to iii) are repeated after at least one hour;
the series of steps i) to iii) are repeated after at least one day;
the method is applied to dispensing of eye drops.

It will be noted that step i) above may be repeated several times before step ii) occurs or an undesired surplus of product appears. In such a scenario, once step i) has been performed, the user releases the pressure on the outer casing of the tube before pressing again to dispense another drop of product and so on until the desired number of drops has been expelled and detached from the tip. The next step ii) can then occur before the stopper has been refitted, or may not occur and the stopper be refitted regardless.

All the aforementioned features set out hereinabove in relation to the device may also be reconsidered here in relation to the method.

Other features and advantages will become apparent during the course of the following description given solely by way of nonlimiting example and made with reference to the attached drawings, in which:

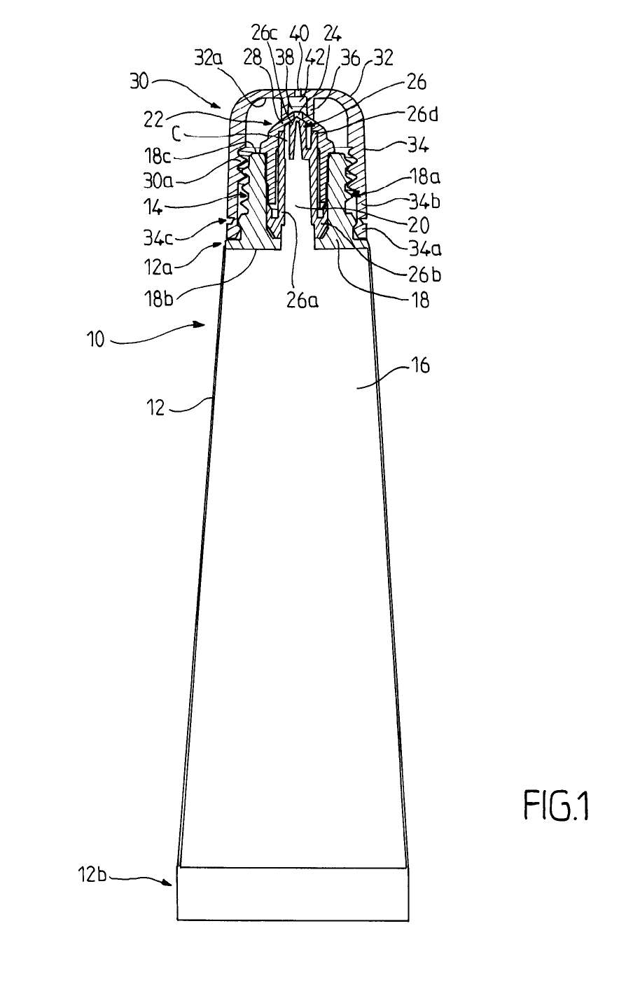

FIG. 1 is a schematic overview of a drop dispensing device according to a first embodiment of the invention;

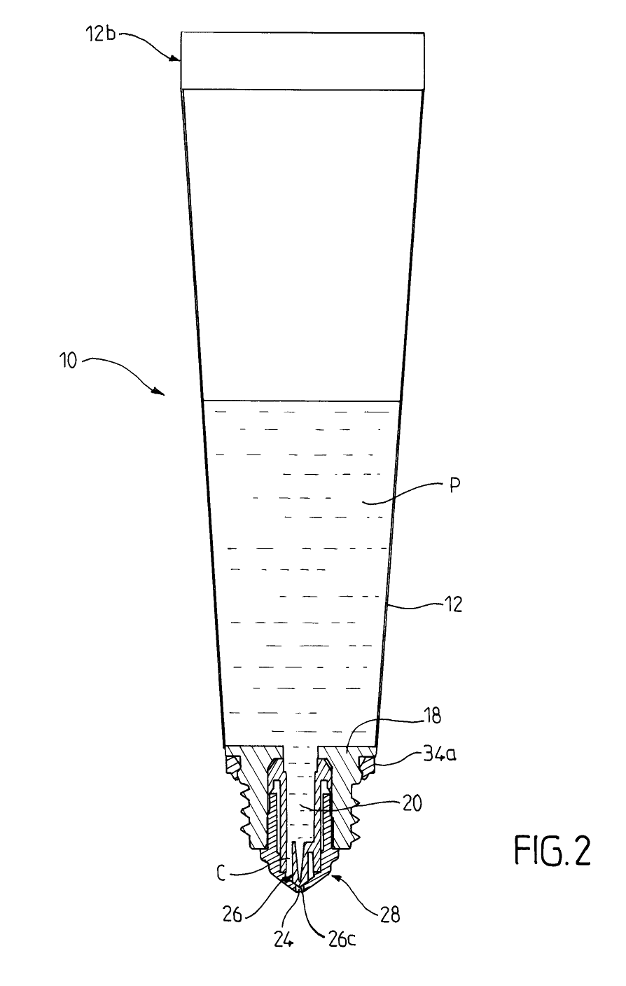

FIG. 2 is a view of the device of FIG. 1 in the inverted position so that it can be used;

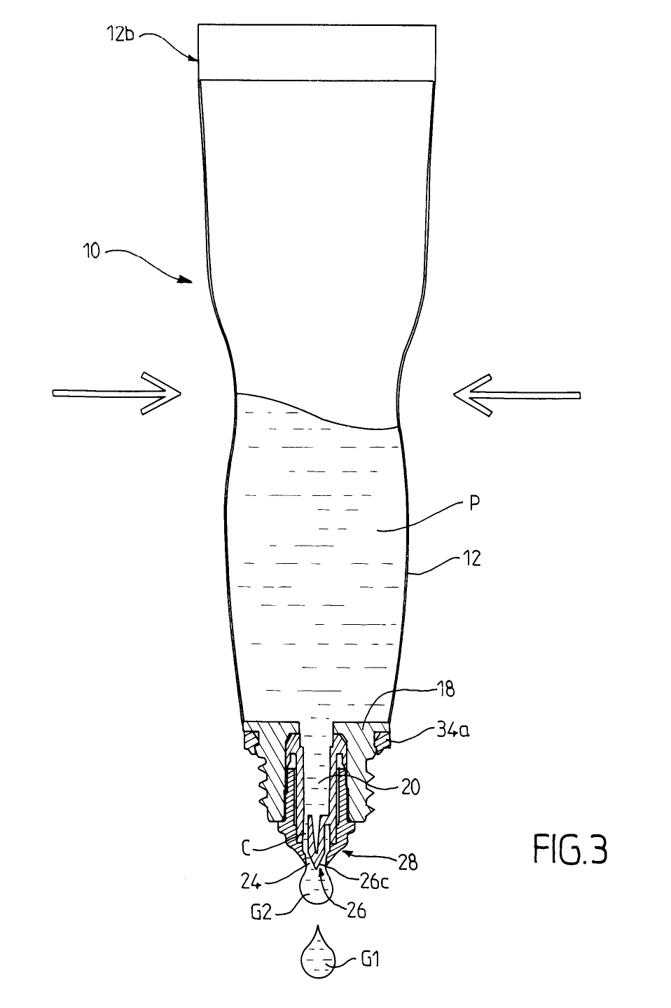

FIG. 3 is a similar view of the device of FIG. 2 in the course of being used;

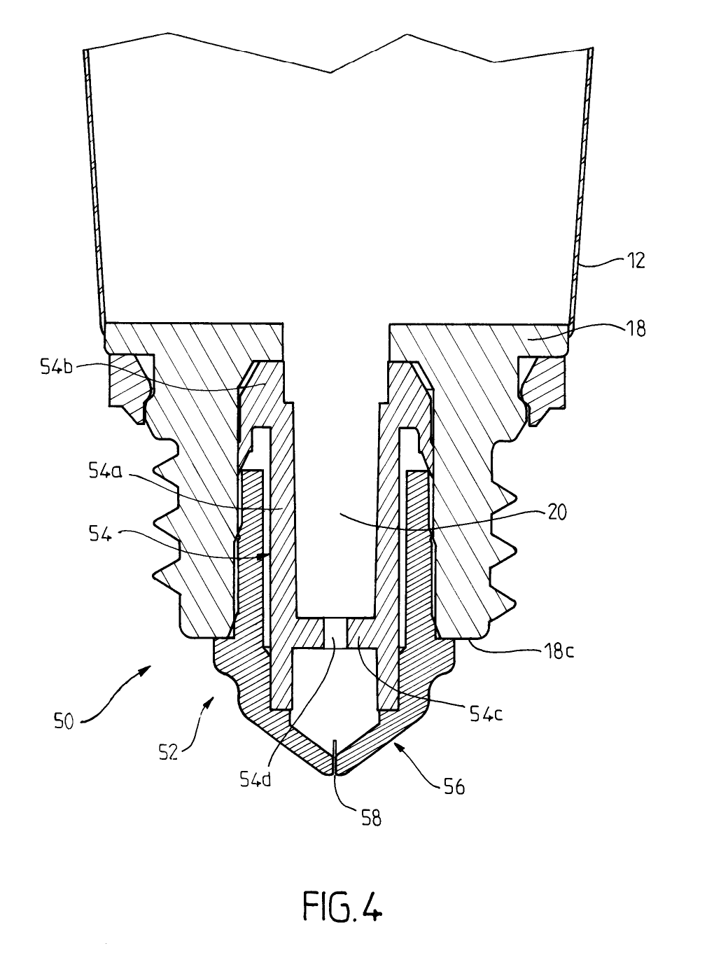

FIG. 4 is a partial schematic view of a drop dispensing device according to a second embodiment of the invention;

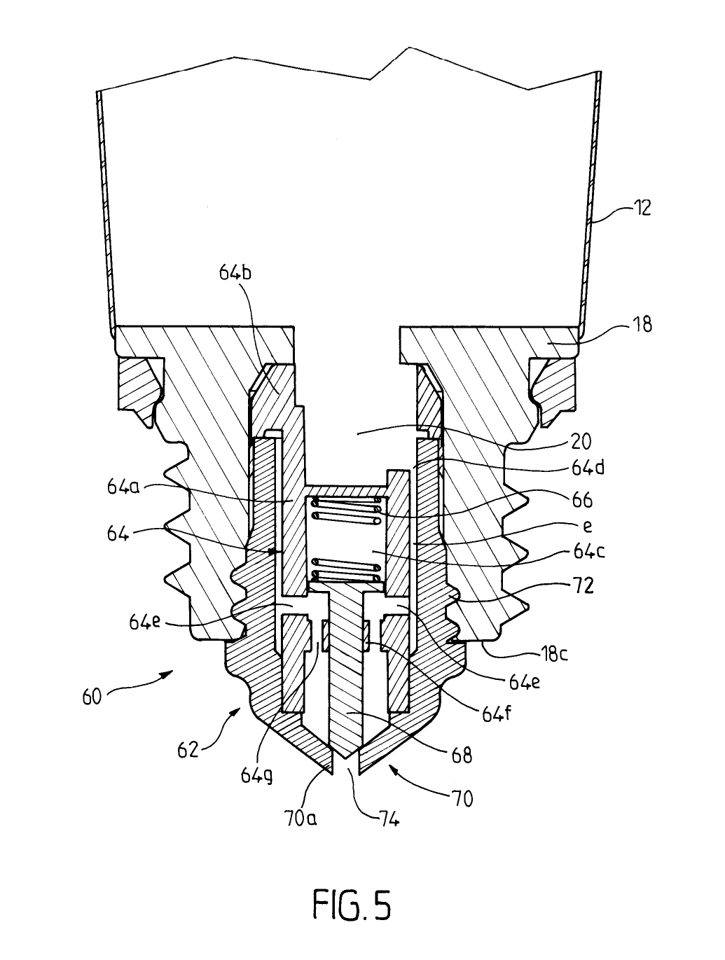

FIG. 5 is a partial schematic view of a drop dispensing device according to a third embodiment of the invention.

As depicted in FIG. 1 and denoted by the general reference 10, a device for dispensing drops of a liquid or semi-liquid product according to a first embodiment of the invention comprises a container 12 in the form of a flexible tube on which a drop-generating tip 14 is mounted. In this embodiment, the product is liquid and is, for example an ophthalmological product. Alternatively, the product may for example be a very fluid cream such as a milk, or even a gel.

In FIG. 1 the tube is arranged vertically in the position for storage, with the tip positioned at the top of the tube. The tube is a conventional commercially available tube.

More particularly, the tube 12 takes the form of a deformable outer casing made for example of polyethylene or polypropylene and define an internal space 16 which contains the product that is to be dispensed (which has not been depicted in FIG. 1).

The tube 12 has two opposite ends, namely a top end 12a in FIG. 1 which is provided with a first opening at which the tip 14 is fixed and a bottom end 12b which forms a tube bottom. During manufacture of the tube, the tube bottom is left open (second opening) so that the tube can be filled with product. Once the product has been introduced into the internal space 16 of the tube, the bottom is sealed, for example by squeezing and welding together the edges of the outer casing delimiting the second opening.

The drop-generating tip 14 comprises a neck-forming part 18 which is fixed at the edges of the first opening (the tip is for example force-fitted into the opening but alternatively could be clip-fastened, screwed in/on, etc.) which, in its central portion, defines a passage 20 for the product that is dispensed.

The neck-forming part 18 on its outer surface has an external screw thread 18a and a substantially cylindrical overall shape extending axially from a widened base 18b fixed to the casing of the tube to a top 18c. The central passage 20 extends axially from the base to the top of the neck 18.

The tip 14 also comprises a valve system 22 arranged partially inside the passage 20 and able, on the one hand, to form a drop of product and expel it from the valve system (when the tube is inverted in the position of use as in FIGS. 2 and 3) following external pressure applied to the outer casing and, on the other hand, to prevent any return of product into the valve system when the external pressure on the casing ceases.

The valve system 22 in general comprises two functional elements:
an orifice 24 through which a drop forms as product passes through the orifice following pressure applied to the outer casing,
means for plugging the orifice 24 which are able to allow the orifice to open following pressure applied to the outer casing and, in the absence of such pressure, to plug the orifice.

The orifice-plugging means comprise two main elements:
a rigid member (e.g. a valve needle) 26 which extends into the passage 20,
a flexible wall or component 28 (which is elastically deformable such as a lip which deforms under pressure; e.g. elastomer wall) arranged around the rigid member 26 and bearing, on the one hand, against the exterior surface of the rigid member 26 and, on the other hand, on the top 18c of the neck 18 and on the internal surface thereof.

The orifice 24 is made in the central portion (arranged along the longitudinal axis of the passage 20) of the flexible wall 28.

The rigid member 26 comprises a first part 26a of substantially cylindrical shape which is mounted inside the passage 20 and rests against the internal surface of the neck 18 via its lower end 26b.

The rigid member also comprises a second part 26c forming a valve needle which is fixed to the upper end 26d of the member and partially inside the latter. The second part 26c extends partially inside the first part 26a and with the internal surface thereof forms an annular duct c for product to flow from the passage 20 as far as the flexible wall 28. The two parts 26a and 26c of the rigid member are in a fixed position relative to one another.

In the rest (non-use) position of FIG. 1, the flexible wall 28 extends over the second part 26c so as to cover it, except in vertical alignment with the orifice 24. In the absence of pressure applied to the outer casing of the tube (FIG. 1) the edges of the flexible wall 28 which delimit the orifice 24 (these edges form something like deformable lips) come into contact with the substantially conical end of the second part 26c. The elasticity of the material of which the flexible wall 28 is made allows sealed contact to be made. The orifice 24 for its part remains open and the tip of the substantially conical end of the second part 26c enters it in part. The valve system described hereinabove is described in greater detail in French patent no. 2 916 657.

As depicted in FIG. 1, the device 10 comprises a protective stopper 30 which is fixed removably to the device so as to cover the drop-generating tip 14 when the device is not being used. In the present embodiment, the stopper 20 on its internal surface comprises a screw thread 30a that complements the screw thread 18a which, through collaboration, will allow the stopper subsequently to be unscrewed and screwed on in relation to the tip.

The protective stopper 30 comprises a substantially planar part 32 (in this instance horizontal) forming a bottom and a skirt-forming part 34 which extends at right angles (in this instance along a vertical axis) from the bottom 32. The screw thread 30a that complements the screw thread 18a is formed on the internal face of the skirt 34.

The bottom 32 has an internal face 32a which faces towards the inside of the stopper and, more particularly, facing the tip 14 when the stopper 30 is fixed on the device (FIG. 1). The bottom 32 in its central zone comprises a cylindrical wall or collar 36 which extends parallel to the skirt 34 (axial direction) away from the internal face 32a. The cylindrical wall 36 delimits a central internal cavity arranged facing the orifice 24. The bottom 32 is pierced at its centre in vertical alignment with the central cavity or housing 38, with a through-hole 40.

An element 42 made of absorbent material (for example made of fibre, felt, foam, for example polyethylene foam, etc.) is housed and held in the bottom of the cavity 38 so as to be in contact with the hole 40 (or very close thereto) and arranged facing the orifice 24.

As illustrated in FIG. 1, the element 42 made of absorbent material is positioned between the hole 40 and the tip 14 and some distance away from the flexible wall 28 and therefore from the orifice 24 so as to avoid any risk of contaminating these. The element 42 for example takes the form of a disc.

As depicted in FIG. 1, when the stopper 30 is mounted fixed on the device, the free end of the wall 36 secured to the bottom 32 rests or presses against the edges of the flexible wall 28 which delimit the orifice 24. This measure thus ensures that the flexible wall 28 held against the second part 26c of the rigid member 26 is locked in position. This then ensures the sealing of the device so equipped.

The skirt 34 of the stopper is made up of two portions: a lower first portion 34a fixed (for example by clip fastening) to a shoulder formed on the exterior surface of the neck 18 of the tip 14, below the screw thread 18a, and an upper second portion 34b forming the skirt and secured to the bottom 32. The two portions 34a and 34b are connected to one another via a thinned zone 34c constituting a mechanically weakened zone that makes it easier for the two portions to be broken apart by unscrewing the upper second portion 34b thereby separating these portions. The first portion 34a constitutes a tamper-evident ring which when present on the device guarantees that the device has not yet been used, when the two portions 34a, 34b of the skirt are still joined together.

It will be noted that the device 10 comprises no pumping means, and notably no pumping means comprising a plunger mounted on a metering pump, for raising the product to the top of the device so that it can be dispensed. With such a pumping means, the user is not in control of the distributed dose of product because this dose is dictated by the pump. The device 10 according to the embodiment of the invention is therefore of simpler design than a device requiring such a pumping means.

The device 10 can thus be used only in an inverted position as depicted in FIGS. 2 and 3.

On first use of the device, the protective stopper 30 is removed (by unscrewing the skirt 34) and the device is inverted as illustrated in FIG. 2. The liquid product P thus occupies that part of the device that is fitted with the tip 14 and enters the passage 20 and the duct c, but is held back by the flexible wall 28 which is still closed around the rigid member 26.

In order to dispense a drop of product P, the user holds the tube between the digits of one hand and presses on the deformable outer casing of the tube 12 as indicated by the arrows in FIG. 3. By deforming the casing in this way, the volume of the internal space 16 containing the product is reduced, thereby causing a rise in pressure of the liquid product in the casing. The pressure of the liquid product inside the tube is therefore proportional to the force applied by the user to the tube. The force applied by the user is, for example, between 200 and 500 g.

The pressurized liquid product presses against the internal face of the flexible wall 28, thus causing the edges of the wall that delimit the orifice 24 to move apart outwards (radially). The orifice 24 moves away from the second part 26c of the rigid member 26 and widens, thus allowing the liquid product to flow through the orifice (cylindrical outlet). By pressing the tube slowly, consistently and with reasonable pressure, the phenomena of adhesion of substances in contact with the product (surface tension) allow the product to be kept suspended from the end of the valve system and thus forming a drop of product. When its weight becomes greater than the resistance generated by the surface tensions that hold the drop on, this drop falls off.

In FIG. 3 a first drop G1 has been formed and expelled from the tip and a second drop G2 in the process of being formed remains “attached” at the end of the valve system. It will be noted that the volume of the drop is defined by a number of parameters which are the shapes, dimensions, surface finishes, materials of the valve system and the actual make up of the product (viscosity, etc.).

A drop of product thus formed corresponds to a dose of product. The user controls his dose because he himself generates the drops whereas in a device fitted with a pumping means it is the pump that controls the dose but does not control the drops.

When the user stops pressing on the tube, the pressure of the liquid product therein drops and, because of the elasticity of the material of which the flexible wall is made, the wall closes again around the rigid member 26 and reverts to its initial shape, as does the orifice 24 (FIGS. 1 and 2). The duct c is thus closed again, preventing any passage of product out of the tip.

When the flexible wall 28 reverts to its initial shape, the edges of the wall which delimit the orifice 24 (deformable lips) close up and thus separate off the drop G2 that is in the process of being formed from the product remaining inside the valve system. The partially formed drop G2 thus remains suspended from the outer end of the valve system in its closed position. As a result, the drop is not aspirated back inside the valve system when pressure on the tube ceases. What happens is that the weight of the partial drop G2 is lower than the surface tension that keeps it attached to (in contact with) the orifice 24 and the edges of the deformable wall. The partial drop G2 is thus suspended from the orifice. This function of preventing the product from returning (preventing contamination) is advantageous because it makes it possible to avoid the need to add preservatives (antioxidants, bactericides, etc.) to the product that is to be dispensed or at the very least to limit the addition of such additives. It is therefore possible for the device to be used again (making it a multi-dose device) to dispense one or more drops of product (according to the desired dose) immediately after or even later in the same day, one or more hours later, or even another day and, for example, repeatedly over the course of time over a predetermined period.

It will be noted that, in order to generate several drops in succession it is possible either to alternate the steps of pressing and releasing the tube or to press against the tube for a prolonged period.

If the drop G2 illustrated in FIG. 3 is not intended to be used, the user inverts the device to place it in the position of FIG. 1 and screws the stopper 30 back on. Alternatively, the stopper can be screwed on while the device is head down.

The element 42 made of absorbent material of the stopper is kept away from the orifice 24 at a distance such that it comes into contact with the drop G2 adhering to the orifice and, through capillarity, absorbs this drop so as to protect the free end of the valve system. This arrangement allows any drop that may have been contaminated by the ambient air to be detached quickly from the end of the drop-generating tip. This then avoids too great a proliferation of bacteria on the dispensing device. It also avoids not dirtying the device when the stopper is refitted. The vent hole 40 in the bottom of the stopper allows the drop of product that has been absorbed by the element 42 made of absorbent material to evaporate. The stopper locks the deformation of the flexible wall 28 (deformable flexible lips) and therefore makes the valve system (and the tip) completely fluid tight: no drop can form (even when the device is inverted as in FIG. 3).

It will also be noted, that when the tube is being filled, the product is introduced into the internal space 16 of the tube leaving a predetermined free volume unoccupied by the product. This predetermined free volume is greater than the volume of product contained in the non-deformable zone of the device. The predetermined volume that is to be left free of product is dependent on the volume of the non-deformable zone. This zone corresponds to the zone of the tube (generally situated near the upper end 12a of the tube and below the base 18b of the neck 18) in which residual product remains trapped at the end of use of the device because of the non-deformable nature of the device in this zone. The predetermined free volume is thus occupied by a gas such as air (or even an inert gas such as nitrogen), which, under the effect of the pressure applied by the user to the outer casing of the tube, drives the product accumulated in this zone towards the passage 20. This measure improves the yield of the device. By way of example, a predetermined air volume representing approximately one third of the volume of the internal space 16 of the tube is left.

The valve system of the tip may alternatively be replaced by a deformable flexible wall in which a slit (orifice) is made and which (in the inverted position) opens under the effect of the external pressure applied to the outer casing of the tube. When the pressure ceases, the slit closes back on itself and separates the drop in the process of being formed from the product remaining in the internal passage of the tip. The tip has no member forming a valve needle as it did in the embodiment of FIGS. 1 to 3. Everything stated hereinabove with regard to the protective stopper and the functionalities thereof (presence of at least one element made of absorbent material distant from the orifice and/or stopper resting via at least part of its internal face against the tip (against the wall surrounding the orifice) and/or through-hole in the wall of the stopper) also applies to this alternative solution which may therefore likewise comprise such a stopper.

A second embodiment of a drop dispensing device 50 is partially illustrated in FIG. 4 in the inverted position and the elements common to those of the first embodiment maintain the same references. Only the tip differs between the two embodiments. The device 50 comprises a drop-generating tip 52 which comprises the neck 18 and a rigid member 54 which is mounted inside the passage 20. The rigid member comprises a first part 54a of substantially cylindrical shape which is mounted inside the passage 20 and rests on the internal surface of the neck 18 via its end 54b which is a lower end when the device is in the rest position (the end 54b is represented uppermost in the figure because the device is in the inverted position for use). The first part 54a extends axially in the passage 20 beyond the neck 18. A transverse partition 54c pierced with a through-hole 54d is formed inside the first part 54a. The tip also comprises a flexible wall or component 56 arranged around the rigid member 54 and which rests, on the one hand, against the exterior surface of the rigid member 54 and, on the other hand, on the top 18c of the neck 18 and on the internal surface of this neck. The flexible component 56 comprises a slit 58 which, in the position of FIG. 4 (the user not pressing on the deformable outer casing of the tube 12 as in FIG. 3), is closed. The slit 58 opens when the user presses on the tube (as in FIG. 3) under the effect of the pressurizing of the product which passes through the hole 54d, accumulates downstream thereof and thus by its weight forces the slit to open and a drop to be generated.

A third embodiment of a drop dispensing device 60 is partially illustrated in FIG. 5 in the inverted position and elements common to those of the first embodiment keep the same references. Only the tip 62 differs from the tip 14 of the first embodiment.

The tip 62 thus comprises a rigid member 64 which is mounted inside the passage 20. The rigid member comprises a first part 64a of substantially cylindrical shape which is mounted inside the passage 20 and rests on the internal surface of the neck 18 via its end 64b which is a lower end when the device is in the rest position (the end 64b is depicted uppermost in the figure because the device is in the inverted position for use). The first part 64a extends axially into the passage 20 beyond the neck 18.

The first part 64a comprises, inside the cylinder that it forms, a chamber 64c housing an elastic means such as a compression spring 66. The rigid member 64 also comprises a mobile rigid component forming a valve needle 68 which is mounted on the elastic means 66 and is capable of sliding axially in the passage 20.

The tip 62 also comprises a component or wall 70 arranged around the rigid member 64 and resting, on the one hand, against the exterior surface of the rigid member 64 and, on the other hand, on the top 18c of the neck 18 and on the internal surface thereof. The component 70 is similar to that of FIGS. 1 to 3. However, it differs therefrom notably in that it is rigid and by the presence of means 72 of connection between the component 70 and internal surface of the neck 18. In this example, these are complementary screw threads but other alternative means of connection are conceivable.

The component 70 is configured at its end with a peripheral edge 70a which delimits a central orifice 74 that is always open. This orifice 74 is plugged by the presence of the mobile valve needle 68 when no external pressure is applied to the tube 12.

The first part 64a is spaced away from the internal surface of the component 70 inside which it is arranged in such a way as to delimit an annular cylindrical space e (the first part 64a is configured to leave an inlet 64d to this space e open near its base). The first part 64a is pierced with radial holes 64e in its cylindrical part so as to place the space e in communication with the internal space at the first part 64a. A pierced transverse partition 64f is arranged inside the cylinder that the first part 64a forms so as to act as a guide to guide the movement of the valve needle 68 and, via the axial holes 64g, allow the product to pass.

When the user presses the tube and pressurizes the product (as in FIG. 3) the product enters the space e, passes through the radial holes 64e, the axial holes 64g and accumulates upstream of the component 70, between this and the base of the valve needle 72. The accumulated pressure of product causes the valve needle to lift into the chamber 64c and uncover the orifice 74. The product is then free, under its own weight, to pass through the orifice 74 (which maintains its initial shape), thereby forming a drop.

According to an alternative form that has not been depicted, the spring 66 is omitted, the valve needle 68 is no longer able to move and only the wall 70 which is flexible widens or contracts according to the pressure or lack of pressure applied to the tube.

According to another alternative form that has not been depicted, the valve system of the tip may, in addition to a deformable flexible wall, the edges of which delimit an orifice that is closed in the position of rest, comprise a mobile rigid member, which, under the action of the pressurized product in the tube and through an appropriate mechanism, forces the orifice to open so as to allow the product to pass.

Any other tip structure capable of forming a drop of liquid or semi-liquid product on the outside of the tip by pressing on the tube and of preventing the product from re-entering the tip when the external pressure on the tube is released (as is the case with the devices described hereinabove) is also suitable for being fixed to a conventional tube with a deformable outer casing.

The embodiments of FIGS. 4 and 5 and alternative forms thereof together with a device provided with any other tip structure as defined hereinabove, may also comprise a protective stopper as defined above (presence of at least one element made of absorbent material distant from the orifice and/or stopper resting via at least part of its internal face against the tip (against the wall surrounding the orifice) and/or through-hole in the wall of the stopper) and the aforementioned advantages relating to the stopper also apply here.

1. Device for dispensing drops of a liquid or semi-liquid product, characterized in that it comprises:
a tube (12) which comprises, on the one hand, a deformable outer casing defining an internal space (16) containing the product and, on the other hand, an opening,
a drop-generating tip (14) which is arranged at the opening and is able, on the one hand, to form and expel a drop (G1, G2) of product from the tip following pressure applied to the outer casing, and which tends to deform it and reduce the volume of the internal space and, on the other hand, to prevent any return of product into the tip when the pressure on the casing ceases, the drop-generating tip (14) comprising an orifice (24) through which a drop forms as the product passes through said orifice as a result of a pressure applied to the outer casing, the orifice (24) being delimited at its periphery by at least one deformable wall (28) which, on deforming, allows a drop to be formed,
a protective stopper (30) which is fixed removably to the device to cover the drop-generating tip (14) when the device is not being used, the protective stopper (30) comprising an internal face (32a) which is directed to face the drop-generating tip (14) when the protective stopper is fixed to the device, the internal face of the protective stopper being provided with at least one element (42) made of absorbent material intended to absorb a drop at least partially formed on the outside of the orifice (24) and which has not detached from this orifice, said at least one element (42) made of absorbent material being distant from the orifice (24).

2. Device according to claim 1, characterized in that the drop-generating tip (14) comprises means of plugging the orifice (24) which are able to allow the orifice to open following a pressure applied to the outer casing and, in the absence of such pressure, to plug the orifice.

3. Device according to claim 1, characterized in that said at least one deformable wall (28) is made of a flexible material.

4. Device according to claim 3, characterized in that said at least one flexible wall (28) is able to close back:
either around a rigid member (26) in the absence of pressure on the outer casing and to move away from the rigid member following pressure applied to the outer casing,
or on the orifice (58) in the absence of pressure on the outer casing.

5. Device according to claim 1, characterized in that it has no means of pumping the product.

6. Device according to claim 1, characterized in that it is able to dispense a drop only when the tube is positioned with the drop-generating tip facing downward.

7. Device according to claim 1, characterized in that the internal face (32a) of the protective stopper is configured to press against said at least one deformable wall (28) and prevent it from it being deformed while the protective stopper is fixed to the device.

8. Device according to claim 1, characterized in that the protective stopper (30) is pierced with a hole (40) open to the outside of the device.

9. Device according to claim 8, characterized in that said at least one element (42) made of absorbent material is arranged between the through-hole (40) and the orifice (24) so as to allow the product absorbed by said element made of absorbent material to evaporate.

10. Device according to claim 1, characterized in that said at least one element (42) made of absorbent material is positioned facing the orifice (24) of the tip.

11. Device according to claim 7, characterized in that the internal face (32a) of the protective stopper is thus configured in a zone which surrounds said at least one element (42) made of absorbent material.

12. Application of the device according to claim 1 to the dispensing of eye drops.

13. Method for dispensing drops of a liquid or semi-liquid product from a device, characterized in that the device comprises:
a tube (12) comprising, on the one hand, a deformable outer casing defining an internal space (16) containing the product and, on the other hand, an opening,
a drop-generating tip (14) which is arranged at the opening and comprises an orifice (24) which is delimited at its periphery by at least one deformable wall (28),
when the tube is positioned with the drop-generating tip facing downwards, the method comprising at least the following steps i) and ii):
i) application of an external pressure to the deformable outer casing of the device so as to deform it and produce the volume of the internal space so as, on the one hand, to form at least one drop of product following deformation of said at least one deformable wall (28) and the product passing through the orifice (24) of the tip and, on the other hand, to expel said at least one drop thus formed from the tip,
ii) ceased application of an external pressure to the deformable outer casing of the device, the product situated outside the orifice (24) of the tip being prevented from returning into the tip as a result of the reverse deformation of said at least one deformable wall (28) and of the at least partial re-closure of the orifice (24), the product situated outside the orifice (24) thus being suspended from the orifice (24),
iii) fitting of a protective stopper onto the device so as to cover the tip, the stopper comprising at least one element (42) made of absorbent material which is distant from the orifice (24) when the stopper is fixed on the device and which, through contact with the product suspended from the orifice (24) on the outside thereof, absorbs said product.

14. Method according to claim 13, characterized in that step i) is repeated over the course of time so as to dispense several drops of product.

15. Method according to claim 13, characterized in that it is applied to the dispensing of eye drops.

16. Device according to claim 2, characterized in that said at least one deformable wall (28) is made of a flexible material.

17. Device according to claim 7, characterized in that the protective stopper (30) is pierced with a hole (40) open to the outside of the device.

18. Device according to claim 17, characterized in that the internal face (32a) of the protective stopper is thus configured in a zone which surrounds said at least one element (42) made of absorbent material.

19. same as 10/7. Device according to claim 7, characterized in that said at least one element (42) made of absorbent material is positioned facing the orifice (24) of the tip.

20. Device according to claim 19, characterized in that the internal face (32a) of the protective stopper is thus configured in a zone which surrounds said at least one element (42) made of absorbent material.
